(19) United States
(12) Patent Application Publication
(10) Pub. No.: US 2008/0206170 A1
(43) Pub. Date: Aug. 28, 2008

(54) Creatine compositions for skin treatment

(76) Inventors: Belinda Tsao Nivaggioli, Atherton, CA (US); Bolko Zu Stolberg, Palo Alto, CA (US); Maisie Wong-Paredes, Redwood City, CA (US)

Correspondence Address: LAHIVE & COCKFIELD, LLP, ONE POST OFFICE SQUARE, BOSTON, MA, 02109, US

(21) Appl. No.: 11/999,994

(22) Filed: Dec. 7, 2007

Related U.S. Application Data
(60) Provisional application No. 60/873,432, filed on Dec. 7, 2006.

(51) Int. Cl. A61K 31/195; A61K 8/40; A61Q 19/00; A61Q 17/04; A61P 17/00; A61P 37/00

(52) U.S. Cl. 424/59; 514/565

(57) ABSTRACT

The present invention relates to the use of creatine compounds, such as, for example, creatine monohydrate, creatine pyruvate and creatine ascorbate, for the treatment of skin.

### RELATED APPLICATIONS

This application claims priority to U.S. Provisional Patent Application No. 60/873,432, filed on Dec. 7, 2006. The aforementioned application is hereby incorporated by reference in its entirety.

### BACKGROUND OF THE INVENTION

The creatine kinase/creatine phosphate energy system is only one component of an elaborate energy-generating system found in tissue with high and fluctuating energy requirements. The components of the creatine energy system include the enzyme creatine kinase, the substrates creatine and creatine phosphate, and the transporter of creatine. The reaction catalyzed by creatine kinase is: MgADP+PCr⁼+H⁺ MgATP⁼+Cr. Some of the functions associated with this system include efficient regeneration of energy in cells with fluctuating and high energy demands, energy transport to different parts of the cell, phosphoryl transfer activity, ion transport regulation, and involvement in signal transduction pathways.

Creatine is a compound which is naturally occurring and is found in mammalian brain and other excitable tissues, such as skeletal muscle, retina and heart. Its phosphorylated form, creatine phosphate, also is found in the same organs and is the product of the creatine kinase reaction utilizing creatine as a substrate. Creatine phosphate is one of the highest energy generating compounds in the cell and creatine is an excellent stimulant of oxidative phosphorylation and high energy production. Creatine has been extensively used by body builders as a means of stimulating energy production in the skeletal muscle. Creatine and creatine phosphate can be synthesized relatively easily and are believed to be non-toxic to mammals. Creatine, creatine phosphate and the enzymes that utilize them as substrates (i.e. the creatine kinases) represent an efficient system for the rapid regeneration of energy. Kaddurah-Daouk et al. (WO 92/08456; WO 90/09192; U.S. Pat. No. 5,321,030; and U.S. Pat. No. 5,324,731) describe methods of inhibiting the growth, transformation and/or metastasis of mammalian cells using related compounds. Examples of compounds described by Kaddurah-Daouk et al. include cyclocreatine, b-guandidino propionic acid, homocyclocreatine, 1-carboxymethyl-2-iminohexahydropyrimidine, guanidino acetate and carbocreatine. These same inventors have also demonstrated the efficacy of such compounds for combating viral infections (U.S. Pat. No. 5,321,030). Elebaly in U.S. Pat. No. 5,091,404 discloses the use of cyclocreatine for restoring functionality in muscle tissue. Cohn in PCT publication No. WO 94/16687 described a method for inhibiting the growth of several tumors using creatine and related compounds. Kaddurah-Daouk et al. (WO 96/14063) reported on the neuroprotective effect of creatine compounds especially against neurodegenerative diseases such as Huntington's, Parkinson's, ALS, Alzheimer's.

Aging involves death of cells or cell dysfunction due to production of free radicals, oxidative damage and energy depletion due to mitochondrial dysfunction. Harman (1988) linked senescence or death to the injurious effects of free radicals arising from the one-electron reduction of oxygen during metabolism. There has been an inverse relationship between auto-oxidation rate in different animal species and life expectancy in the same species (Cutler 1985; Sohal 1995). Mitochondria are the major source of oxygen radicals through the respiratory chain and are also deeply affected by reactive oxygen species (ROS), resulting in serious risks to their function. Mitochondrial dysfunction could result in defects in electron transport, oxidative phosphorylation and energy production resulting in cell damage and ultimately cell death.

### SUMMARY OF THE INVENTION

In one embodiment, the invention pertains, at least in part, to a method for treating uneven pigmentation in a subject's skin by administering to a subject an amount of a creatine compound effective to modulate tyrosinase, in which the amount of the creatine compound is at least between about 0.0001 and 10% by weight.

In another embodiment, the invention pertains, at least in part, to a method for quenching free radicals in a subject's skin, by administering to a subject an amount of a creatine compound effective to provide an antioxidant effect, in which the amount of the creatine compound is at least between about 0.0001 and 10% by weight.

In yet another embodiment, the invention pertains, at least in part, to a method for preserving a subject's skin, by administering to a subject an amount of a creatine compound effective to inhibit lipid peroxidation, in which the amount of the creatine compound is at least between about 0.0001 and 10% by weight.

In a further embodiment, the invention pertains, at least in part, to a method for treating aging of a subject's skin, by administering to a subject an amount of a creatine compound effective to modulate mitochondrial metabolism, in which the amount of the creatine compound is at least between about 0.0001 and 10% by weight.

In one embodiment, the invention pertains, at least in part, to a method of treating a subject's skin for UV irradiation stress by administering an effective amount of a creatine compound to the subject, wherein said effective amount of a creatine compound is a least between about 0.0001 and 10% by weight.

In one embodiment, the invention pertains, at least in part, to a method of modulating carcinogenic stress in a subject's skin, by administering an effective amount of a creatine compound to a subject.

In yet another embodiment, the invention pertains, at least in part, to a method for modulating collagen levels in a subject's skin, comprising administering to said subject of a at least between about 0.0001 and 10% by weight of a creatine compound, such that collagen levels in said subject's skin are modulated.

In a further embodiment, the invention pertains, at least in part, to a method of treating inflammation in a subject, by administering an amount of a creatine compound effective to modulate metalloproteinase.

In one embodiment, the invention pertains, at least in part, to a cosmetic composition comprising an effective amount of a creatine compound, a cosmetically acceptable carrier, and one or more cosmetic adjuvants, in which the effective amount of the creatine compound is at least between about 0.0001 and 10% by weight.

In yet another embodiment, the invention further pertains, at least in part, to a packaged composition comprising an effective amount of a creatine compound, a cosmetically acceptable carrier, and one or more cosmetic adjuvants, in which the effective amount of the creatine compound is at least between about 0.0001 and 10% by weight.

### BRIEF DESCRIPTION OF THE DRAWINGS

FIG. 1 is a graph illustrating the concentration-dependent effect of creatine ascorbate (♦), creatine monohydrate (▪), creatine pyruvate (▴), ascorbic acid (×) and magnesium ascorbyl phosphate (•) on the inhibition of lipid peroxidation.

FIG. 2 is a chart illustrating the concentration-dependent effect of freshly prepared creatine ascorbate (CA), creatine monohydrate (CM), creatine pyruvate (CP), ascorbic acid (AA) and magnesium ascorbyl phosphate (MAP) solutions on the stimulation of type I collagen levels in human dermal fibroblasts, compared to the control (—).

FIG. 3 is a chart illustrating the concentration-dependent effect of preincubated creatine ascorbate (CA), creatine monohydrate (CM), creatine pyruvate (CP), ascorbic acid (AA) and magnesium ascorbyl phosphate (MAP) solutions on the stimulation of type I collagen levels in human dermal fibroblasts, compared to the control (—).

FIG. 4 is a chart illustrating the concentration-dependent effect of freshly prepared creatine ascorbate (CA), creatine monohydrate (CM), creatine pyruvate (CP), ascorbic acid (AA) and magnesium ascorbyl phosphate (MAP) solutions on the stimulation of mitochondrial metabolism in human dermal fibroblasts, compared to the control (—).

FIG. 5 is a chart illustrating the concentration-dependent effect of preincubated creatine ascorbate (CA), creatine monohydrate (CM), creatine pyruvate (CP), ascorbic acid (AA) and magnesium ascorbyl phosphate (MAP) solutions on the stimulation of type I mitochondrial metabolism in human dermal fibroblasts, compared with the control (—).

FIG. 6 is a graph illustrating the relative fluorescence intensity of creatine ascorbate (▪), creatine monohydrate (▴), creatine pyruvate (×), ascorbic acid (♦) and magnesium ascorbyl phosphate (•) over 60 minutes.

FIG. 7 is a graph illustrating the time dependant effect of UVB irradiation at 0.15 mW/cm² on human dermal fibroblast proliferation.

FIG. 8 is a chart illustrating the effect of creatine monohydrate (CM Irr), creatine ascorbate (CA Irr), creatine pyruvate (CP Ir), ascorbic acid (AA Irr) and magnesium ascorbyl phosphate (MAP Irr) on p53 expression induced by UVB irradiation of human dermal fibroblasts compared to non-irradiated cells and irradiated control cells.

### DETAILED DESCRIPTION OF THE INVENTION

The methods of the present invention generally comprise administering to a subject an effective amount of a creatine compound or compounds.

The term “creatine compound” includes creatine, derivatives of creatine, creatine-ligand compounds and pharmaceutically acceptable salts of creatine. Examples of derivatives of creatine include cyclocreatine, creatine phosphate, cyclocreatine phosphate, etc. Examples of creatine derivatives are described in U.S. Pat. No. 6,242,491 B1, incorporated herein by reference.

Creatine (also known as N-(aminoiminomethyl)-N-methylglycine; methylglycosamine or N-methyl-guanido acetic acid) is a compound of formula (I):

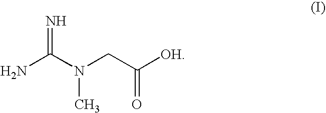

In a further embodiment, the creatine compound is creatine ascorbate, creatine pyruvate, or creatine monohydrate.

In a further embodiment, the creatine compound is a creatine-ligand compound, wherein the creatine-ligand compound has a ratio of between about 1:1 creatine to ligand and about 10:1 creatine to ligand. The term “ligand” includes a compound in which creatine is bound to another atom or molecule through covalent or electrostatic interactions. The ratio of creatine to the ligand can be, for example, about a 1:1 ratio, about a 2:1 ratio, about a 3:1 ratio, about a 4:1 ratio, about a 5:1 ratio, about a 6:1 ratio, about a 7:1 ratio, about an 8:1 ratio, about a 9:1 ratio or about a 10:1 ratio. The ratio of creatine to the ligand can also be any ratio in which creatine is bound to the ligand through covalent or electrostatic interactions. In one embodiment, the creatine-ligand compound has a ratio of between about 3:1 creatine to ligand and about 6:1 creatine to ligand.

In one embodiment, the ligand is an amino acid. The term “amino acid” includes any molecule that contains both an amino and a carboxylic acid functionality and includes standard and non-standard amino acids. Standard amino acids include, for example, leucine, proline, alanine, valine, glycine, serine, asparagine, glutamine, aspartic acid, glutamic acid, methionine, tryptophan, phenylalanine, isoleucine, threonine, cysteine, tyrosine, histidine, lysine and arginine. Non-standard amino acids include all other amino acids, for example, 5-hydroxylysine, 4-hydroxyproline, thyroxine, 3-methylhistadine, ε-N-methyllysine, ε-N,N,N-trimethyllysine, aminoadipic acid, γ-carboxyglutamic acid, pyroglutamic acid, phosphoserine, phosphotyrosine, N-methylarginine, N-acetyllysine, sarcosine, γ-aminobutyric acid, betaine, β-alanine, azaserine, homoserine, lanthionine, homocysteine, phenylserine, chloramphenicol, cycloserine, epinephrine, histamine, serotonin, penicillamine, ornithine, citrulline and the like.

In one embodiment, the ligand is a water-soluble vitamin. The term “water-soluble vitamin” includes those vitamins which dissolve easily in water, such as vitamin C (ascorbic acid) and the B-complex vitamins. The B-complex vitamins may include vitamin B₁ (thiamine), B₂ (riboflavin), B₃ (niacin), B₅ (pantothenic acid), B₆ (pyridoxine), B₇ (biotin), B₉ (folic acid), and B₁₂ (cyanocobalamin).

In another embodiment, the ligand is selected from the group consisting of cinnamate, lactate, glycolate, malate, mandelate, ascorbate, phytate, citrate, hydroxycitrate, aleurate, salicylate and hyaluronate. In one particular embodiment, the ligand is ascorbate.

The term “subject” includes living organisms, such as humans, dogs, cats, horses, goats, cows, pigs, rodents, monkeys, gorillas, bears, chimpanzees and cattle. The term “subject” further is intended to include transgenic species.

In one embodiment, the invention pertains to a method for modulating the melanin synthesis pathway. In humans, melanin is found in skin, hair, the pigmented epithelium underlying the retina, the medulla and zona reticularis of the adrenal gland, the stria vascularis of the inner ear, and in pigment bearing neurons of certain deep brain nuclei such as the locus ceruleus and the substantia nigra. Melanin is believed to be the primary determinant of human skin color. Dermal melanin is produced by melanocytes, which are found in the stratum basale of the epidermis. Although human beings generally possess a similar concentration of melanocytes in their skin, the melanocytes in some individuals and ethnic groups more frequently or less frequently express the melanin-producing genes, thereby conferring a greater or lesser concentration of skin melanin. Some individual animals and humans have no or very little melanin in their bodies, which is a condition known as albinism.

Because melanin is an aggregate of smaller component molecules, there are a number of different types of melanin with differing proportions and bonding patterns of these component molecules. Both pheomelanin and eumelanin are found in human skin and hair, but eumelanin is the most abundant melanin in humans, as well as the form most likely to be deficient in albinism. The precise nature of eumelanin's molecular structure is the object of study. Eumelanin is found in hair and skin, and colors hair grey, black, yellow, and brown. In humans, it is more abundant in peoples with dark skin. There are two different types of eumelanin, which are distinguished from each other by their pattern of polymer bonds. The two types are black eumelanin and brown eumelanin. A small amount of black eumelanin in the absence of other pigments causes grey color. A small amount of brown eumelanin in the absence of other pigments causes yellow (blond) color. Pheomelanin is also found in hair and skin and is more abundant in fair-skinned humans. Pheomelanin imparts a pink to red hue and, thus, is found in particularly large quantities in red hair. Pheomelanin is particularly concentrated in the lips, nipples, glans of the penis, and vagina. Pheomelanin also may become carcinogenic when exposed to the ultraviolet rays of the sun. Chemically, pheomelanin differs from eumelanin in that its oligomer-structure incorporates the amino acid L-cysteine, as well as DHI and DHICA units.

The first step in the biochemical synthesis of the components of melanin (both eumelanins and pheomelanis) in the body is the tyrosinase-mediated transformation of tyrosine to DOPA (3,4-dihydroxy-L-phenylalanine) and dopaquinone. Dopaquinone can then combine with cysteine to form either 5-S-cysteinyldopa or 2-S-cysteinyldopa, which, via a benzothiazine intermediate, forms pheomelanin. Alternatively, dopaquinone can be converted to leucodopachrome and dopachrome, which can subsequently be converted to either 5,6-dihydroxyindole-2-carboxylic acid or 5,6-dihydroxyindole. These two compounds are then converted into quinine, followed by the production of eumelanin.

The language “melanin synthesis pathway” includes any biochemical pathway for the synthesis and/or upregulation of any of the components of melanin in the body, as described above. The language “modulating the melanin synthesis pathway” includes adjusting the melanin synthesis pathway or keeping the melanin synthesis pathway in proper measure or proportion. In one embodiment, the language “modulating the melanin synthesis pathway” includes upregulating the melanin synthesis pathway or any of the components of the melanin synthesis pathway. In another embodiment, the language “modulating the melanin synthesis pathway” includes inhibiting the melanin synthesis pathway or any of the components of the melanin synthesis pathway.

Skin disorders that may be caused by a disturbance of the melanin synthesis pathway include, for example, albinism and uneven pigmentation (e.g., age spots, liver spots, freckles, moles, etc.) and skin cancer (e.g., melanoma).

The language “treating uneven pigmentation” includes the prevention, alleviation or amelioration of one or more symptoms of uneven pigmentation. For example, treating uneven pigmentation may refer to the fading or disappearance of the uneven pigmentation.

In one embodiment, the invention pertains to the treatment of uneven pigmentation in the skin of a subject by administering to the subject an amount of a creatine compound effective to modulate tyrosinase (e.g., the conversion of tyrosine to DOPA and dopaquinone). The language “an amount of a creatine compound effective to modulate tyrosinase” includes the amount of a creatine compound necessary to upregulate or inhibit tyrosinase activity.

In one embodiment, the amount of creatine compound effective to modulate tyrosinase is at least between about 0.0001% to 10% by weight. More particularly, the amount of the creatine compound necessary to modulate tyrosinase may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.005%, 0.006% 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1%, 2%, 3%, 4%, 5%, 6%, 7%, 8%, 9%, and 10% by weight, or a range thereof. In a further embodiment, the amount of creatine compound effective to modulate tyrosinase is at least between about 0.001% and 1% by weight or between about 0.01% and 0.5% by weight.

In one embodiment, the creatine compound inhibits tyrosinase. The creatine compound may inhibit tyrosinase by about, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% 64%, 65%, 66%, 67%, 68%, 69%, 70%, 71%, 73%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or a range thereof.

In a one particular embodiment, the creatine compounds, such as creatine ascorbate and creatine pyruvate, inhibit tyrosinase by at least about 25% at a concentration of about 5 μg/mL, at least about 100% at a concentration of about 500 μg/mL, or by at least about 10% at a concentration of about 50 μg/mL. The inhibition of tyrosinase can be determined using the methods described in Example 7.

In one embodiment, the invention pertains to a method of treating the skin of a subject for oxidative stress. The language “oxidative stress” includes the imbalance between free radicals and antioxidants, which causes damage to the cells of a subject. Free radicals are atomic or molecular species with unpaired electrons on an otherwise open shell configuration. These unpaired electrons are highly reactive and the amount of free radicals typically increases during environmental stress. Typically, cells are able to defend themselves against free radical damage through the use of enzymes such as superoxide dismutases and catalases.

The effects of free radicals on cell metabolism have been well documented in a variety of species, and include roles in programmed cell death, apoptosis, cancer and inflammatory responses. In addition, free radicals play an important role in the death and regeneration of skin cells.

In one particular embodiment, the invention pertains to a method for quenching free radicals in a subject's skin by administering to the subject an amount of a creatine compound effective to provide an antioxidant effect. The language “quenching free radicals,” includes the suppression of free radicals, inhibition of the generation of free radicals or the treating the damage caused by free radicals. The language “antioxidant effect” includes the effect of quenching free radicals (e.g., the effect of suppressing free radicals, the effect of inhibiting the generation of free radicals or the effect of treating the damage caused by free radicals).

The language “an amount of a creatine compound effective provide an antioxidant effect” includes the amount of a creatine compound necessary to quench at least a portion of the free radicals (e.g., suppression of free radicals, inhibition of the generation of free radicals or the treating the damage caused by free radicals), preferably most, if not all, of the free radicals in the subject's skin. The antioxidant effect of the creatine compounds of the invention can be determined using the method described in Example 4.

In one embodiment, the amount of creatine compound effective to quench free radicals is at least between about 0.0001% to 10% by weight. More particularly, the amount of the creatine compound necessary to modulate tyrosinase may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.005%, 0.006% 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1%, 2%, 3%, 4%, 5%, 6%, 7%, 8%, 9%, and 10% by weight, or a range thereof. In a further embodiment, the amount of creatine compound effective to quench free radicals is at least between about 0.001% and 1% by weight or between about 0.01% and 0.5% by weight.

One method for determining the antioxidant effect of compound is measuring the oxygen radical absorbance capacity (ORAC) of that compound (see Example 4). The units of the ORAC are micromole Trolox equivalents (TE) per gram of compound. The higher the Trolox equivalents, the greater the capacity of the compound to absorb free radicals. In one embodiment, the creatine compound exhibits at least about 5300 μM Trolox equivalents/gram. In another embodiment, the creatine compound exhibits a larger ORAC than magnesium ascorbyl phosphate. In yet another embodiment, the creatine compound exhibits an antioxidant activity greater than magnesium ascorbyl phosphate. In yet another embodiment, the creatine compound is creatine ascorbate.

In one embodiment, the invention pertains to a method for preserving the skin of a subject by administering to a subject an amount of a creatine compound effective to inhibit oxidation of the skin. In another embodiment, the invention pertains to a method for preserving the skin of a subject by administering to a subject an amount of a creatine compound effective to inhibit lipid peroxidation. The language “preserving the skin” includes maintaining the skin or protecting the skin from harm.

The term “lipid peroxidation” includes the oxidative degradation of lipids in cell membranes, resulting in cell damage.

The language “an amount of a creatine compound effective to inhibit lipid peroxidation” includes the amount of a creatine compound necessary to inhibit lipid peroxidation. The inhibition of lipid peroxidation can be determined using the method described in Example 1.

In one embodiment, the amount of creatine compound effective to inhibit lipid peroxidation is at least between about 0.0001% to 10% by weight. More particularly, the amount of the creatine compound necessary to inhibit lipid peroxidation may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.005%, 0.006% 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1%, 2%, 3%, 4%, 5%, 6%, 7%, 8%, 9%, and 10% by weight, or a range thereof. In a further embodiment, the amount of creatine compound effective to inhibit lipid peroxidation is at least between about 0.001% and 1% by weight or between about 0.01% and 0.5% by weight.

In another embodiment, the creatine compound may inhibit lipid peroxidation by about, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% 64%, 65%, 66%, 67%, 68%, 69%, 70%, 71%, 73%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or a range thereof. In one particular embodiment, the creatine compounds, such as creatine ascorbate, inhibit lipid peroxidation by at least about 20% at a concentration of about 500 μg/mL.

Mitochondria play an important role in many metabolic tasks, such as, for example apoptosis-programmed cell death, energy conversion, cellular proliferation and regulation of the cellular redox state, and regulation of mitochondrial metabolism is an important method of treating aging of the skin.

In one embodiment, the invention pertains to a method for treating aging in the skin of a subject by administering to a subject an amount of a creatine compound effective to modulate mitochondrial metabolism. The language “modulate mitochondrial metabolism” includes adjusting the mitochondrial metabolism or keeping the mitochondrial metabolism in proper measure or proportion. In one embodiment, modulating mitochondrial metabolism includes inhibiting mitochondrial metabolism. In another embodiment, modulating mitochondrial metabolism includes stimulating or increasing mitochondrial metabolism.

The language “an amount of a creatine compound effective to modulate mitochondrial metabolism” includes the amount of a creatine compound necessary to inhibit, increase or stimulate mitochondrial metabolism.

In one embodiment, the amount of creatine compound effective to modulate mitochondrial metabolism is at least between about 0.0001% to 10% by weight. More particularly, the amount of the creatine compound necessary to modulate mitochondrial metabolism may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.005%, 0.006% 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1%, 2%, 3%, 4%, 5%, 6%, 7%, 8%, 9%, and 10% by weight, or a range thereof. In a further embodiment, the amount of creatine compound effective to modulate mitochondrial metabolism is at least between about 0.001% and 1% by weight or between about 0.01% and 0.5% by weight.

In another embodiment, the creatine compound may modulate mitochondrial metabolism by about, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% 64%, 65%, 66%, 67%, 68%, 69%, 70%, 71%, 73%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or a range thereof.

In one particular embodiment, the creatine compound stimulates mitochondrial metabolism. In another embodiment, the creatine compounds, such as creatine monohydrate, stimulate mitochondrial metabolism by at least about 15% at a concentration of about 100 μg/mL or by at least about 25% at a concentration of about 1000 μg/mL of the creatine compound. The stimulation of mitochondrial metabolism can be measured using the method described in Example 3.

In one embodiment, the invention pertains to a method of treating UV irradiation stress in a subject's skin by administering an effective amount of a creatine compound to said subject. The term “treating UV irradiation” includes preventing UV radiation or ameliorating the effects of UV radiation. UV radiation can refer to both UVA radiation and UVB radiation.

In another embodiment, the invention pertains to a method of modulating carcinogenic stress in a subject's skin by administering an effective amount of a creatine compound to the subject. The term “modulating carcinogenic stress” includes the inhibition of carcinogenic stress or protection against carcinogenic stress. The language “carcinogen stress” includes any stimulus or circumstance that may induce or cause cancer, such as skin cancer. Examples of stimuli include carcinogenic chemicals, environmental factors and aging. In one embodiment, the stimulus is not sun radiation, UVA, UVB, or UVC radiation. In one embodiment, the cancer may be, for example, basal cell carcinoma, squamous cell carcinoma, malignant melanoma, dermatofibrosarcoma protuberans, Merkel cell carcinoma or Kaposi's sarcoma.

In one embodiment, the creatine compound for modulating carcinogenic stress is creatine monohydrate, creatine pyruvate or creatine ascorbate.

The term “effective amount of a creatine compound” includes the amount of a creatine compound necessary to alleviate, prevent or ameliorate one or more symptoms that the administration of the creatine compound is attempting to treat.

In one embodiment, an effective amount of creatine compound is at least between about 0.0001% to 10% by weight. More particularly, an effective amount of creatine compound may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.005%, 0.006% 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1%, 2%, 3%, 4%, 5%, 6%, 7%, 8%, 9%, and 10% by weight, or a range thereof. In a further embodiment, the effective amount of creatine compound is at least between about 0.001% and 1% by weight or between about 0.01% and 0.5% by weight.

In another embodiment, the invention pertains to a method for treating photodamage and aging in a subject's skin by administering to a subject an amount of a creatine compound effective to modulate collagen levels. The term “photodamage” and “photoaging” may be used to describe chronic changes in the appearance and function of the skin caused by repeated sun exposure rather than by the passage of time (the latter called intrinsic or chronologic aging). The term “acute photodamage” includes sunburn. Photodamage may also be called dermatoheliosis. Symptoms of photodamage include, for example, skin changes of fine and coarse wrinkles, roughness, laxity, mottled pigmentation, actinic lentigines, actinic keratoses, leathery texture/coarseness, scaling/xerosis, sallowness, and telangiectasia, as well as cancer. In addition, environmental factors, such as cigarette smoking, may cause changes in the skin associated with aging. The language “treating photodamage or aging” includes the alleviation, amelioration or prevention of one or more symptoms of photodamage or aging of the skin.

Collagen is the main protein of connective tissue in animals and the most abundant protein in mammals, making up about 25% of the total protein content. It is a long, fibrous structural protein whose function is quite different from those of globular proteins such as enzymes. Strong, tough bundles of collagen called collagen fibers are a major component of the extracellular matrix that supports most tissues and gives cells structure from the outside, but collagen is also found inside certain cells. Collagen has great tensile strength, and is the main component of cartilage, ligaments, tendons, bone and teeth. Along with soft keratin, it is responsible for skin strength and elasticity, and its degradation leads to wrinkles that accompany aging

The language “modulate collagen levels” includes the adjusting collagen levels or keeping collagen levels in proper measure or proportion. In one embodiment, the collagen levels are increased, elevated or stimulated. The language “an amount of a creatine compound effective to modulate collagen levels” includes the amount of a creatine compound necessary to increase, elevate of stimulate collagen levels.

In one embodiment, the amount of a creatine compound effective to modulate collagen levels is at least between about 0.0001% to 10% by weight. More particularly, the amount of a creatine compound effective to modulate collagen levels may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.005%, 0.006% 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1%, 2%, 3%, 4%, 5%, 6%, 7%, 8%, 9%, and 10% by weight, or a range thereof. In a further embodiment, the amount of a creatine compound effective to modulate collagen levels is at least between about 0.001% and 1% by weight or between about 0.01% and 0.5% by weight.

In another embodiment, the creatine compound may modulate collagen levels by about, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% 64%, 65%, 66%, 67%, 68%, 69%, 70%, 71%, 73%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or a range thereof. In one embodiment, the creatine compound stimulates collagen levels. For example, it has been shown that certain creatine compounds, such as creatine ascorbate and creatine pyruvate, stimulate collagen levels by at least about 50% at concentration of about 10 μg/mL.

In another example, the creatine compounds, such as creatine pyruvate, stimulate collagen levels by at least about 20% at a concentration of about 1 μg/mL. Stimulation of collagen levels can be determined by using the assay described in Example 2.

In one embodiment, the invention pertains to a method of treating inflammation in a subject by administering an amount of a creatine compound effective to modulate metalloproteinase. Inflammation is a complicated biochemical response of the immune system to infection or irritation and is characterized by redness, heat, swelling and pain.

Metalloproteinases (or metalloproteases) are a family of enzymes from the group of proteinases. There are two subgroups of metalloproteinases: metallocarboxypeptidases and metalloendopeptidases. Proteinases can be divided into four families if characterized by the nature of the most prominent functional group in their active site: serine, cysteine, aspartic and metalloproteinases. Metalloproteinases bind a metal ion such as Zn²⁺ or Ca²⁺ in their active site. Important metalloproteinases are the bacterial enzyme thermolysin (which is a metalloendopeptidase), the digestive enzymes carboxypeptidase A or B (which are metallocarboxypeptidases), matrix metalloproteinases (MMP, also metalloendopeptidases) and collagenases. Collagenases are a type of metalloproteinases that break down the peptide bonds in collagen. MMPs play an important role in tumor metastasis, embryonic development and wound healing.

The language “modulate metalloproteinase” includes inhibiting or stimulating the metalloproteinase activity. In one embodiment, the metalloproteinase is inhibited.

In one embodiment, the amount of a creatine compound effective to modulate metalloproteinase is at least between about 0.0001% to 10% by weight. More particularly, the amount of a creatine compound effective to modulate metalloproteinase may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.005%, 0.006% 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1%, 2%, 3%, 4%, 5%, 6%, 7%, 8%, 9%, and 10% by weight, or a range thereof. In a further embodiment, the amount of a creatine compound effective to modulate metalloproteinase is at least between about 0.001% and 1% by weight or between about 0.01% and 0.5% by weight.

In another embodiment, the creatine compound may modulate metalloproteinase by about, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% 64%, 65%, 66%, 67%, 68%, 69%, 70%, 71%, 73%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or a range thereof.

In one embodiment, certain creatine compounds, such as creatine ascorbate, inhibit metalloproteinase by at least about 10% at a concentration of about 1 mg/mL. Methods for the determination of the inhibition of metalloproteinase can be found in Example 6.

In another embodiment, the creatine compound inhibits metalloproteinase by at least about 25% at a concentration of about 1 mg/mL of said creatine compound. In a further embodiment, the creatine pyruvate.

The pharmaceutical compositions of the present invention may be made into a wide variety of product types, including, for example, liquids, solids, gelatin capsules, lotions, creams, mousses, aerosols and non-aerosol sprays, gels, emulsions, solutions, ointments, patches or medicated pads.

In one embodiment, the creatine compound is administered topically or orally. The term “topical administration” includes methods of delivery such as laying on or spreading on the skin. It involves any form of administration which involves the skin. Examples of compositions suitable for topical administration, include but are not limited to, ointments, lotions, creams, cosmetic formulations, and skin cleansing formulations. Additional examples include aerosols, solids (such as bar soaps) and gels.

The term “pharmaceutically acceptable” includes drugs, medicaments or inert ingredients which are suitable for use in contact with the tissues of humans and lower animals without undue toxicity, incompatibility, instability, irritation, allergic response, and the like, commensurate with a reasonable benefit/risk ratio. The term also encompasses cosmetically acceptable ingredients.

The language “effective amount” is intended to include the amount of the creatine compound sufficient to prevent, ameliorate or alleviate one or more symptom that the administration of the creatine compound is attempt to treat. An effective amount can be determined on an individual basis and will be based, at least in part, on consideration of the severity of the symptoms to be treated and the activity of the specific analog selected if an analog is being used. Further, the effective amounts of the creatine compound may vary according to the age of the subject being treated. Thus, an effective amount of the creatine compound can be determined by one of ordinary skill in the art employing such factors as described above using no more than routine experimentation in health care management.

The phrase “pharmaceutically acceptable carrier” includes a pharmaceutically acceptable material, composition or vehicle, such as a liquid or solid filler, diluent, excipient, solvent or encapsulating material, involved in carrying or transporting a compound(s) of the present invention within or to the subject such that it can performs its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; fruit acids, pyrogen-free water; isotonic saline; Ringer's solution; ethyl alcohol; phosphate buffer solutions; and other non-toxic compatible substances employed in pharmaceutical formulations.

The topical pharmaceutical compositions of the present invention may be made into a wide variety of product types. These include, but are not limited to solutions, lotions, creams, beach products, gels, sticks, sprays, pads, ointments, pastes, mousses and cosmetics. These product types may comprise several types of carrier systems including, but not limited to solutions, emulsions, gels and solids.

The topical pharmaceutical compositions of the present invention formulated as solutions typically include a pharmaceutically-acceptable aqueous or organic solvent. The terms “pharmaceutically-acceptable aqueous solvent” and “pharmaceutically-acceptable organic solvent” refer to a solvent which is capable of having dispersed or dissolved therein the active compound, and possesses acceptable safety properties (e.g. irritation and sensitization characteristics). Water is a typical aqueous solvent. Examples of suitable organic solvents include: propylene glycol, butylene glycol, polyethylene glycol (200-600), polypropylene glycol (425-2025), glycerol, 1,2,4-butanetriol, sorbitol esters, 1,2,-6-hexanetriol, ethanol, isopropanol, butanediol, and mixtures thereof. Preferably, these solutions contain from about 0.0001% to about 10% of the of the active compound (e.g., a creatine compound), more preferably from about 0.001% to about 1%, and more preferably from about 0.01% to about 0.5%; and from about 1% to about 90% of an acceptable aqueous or organic solvent, more preferably from about 1% to about 40%.

If the topical pharmaceutical compositions of the present invention are formulated as an aerosol and applied to the skin as a spray-on, a propellant is added to a solution composition. A more complete disclosure of propellants useful herein can be found in Sagarin, Cosmetics Science and Technology, 2nd Edition, Vol. 2, pp. 443-465 (1972).

Topical pharmaceutical compositions of the present invention may be formulated as a solution comprising an emollient. An example of a composition formulated in this way would be a sunscreen-containing product. Preferably, such compositions contain from about 0.0001% to about 10% of the active compound (e.g., a creatine compound), more preferably from about 0.001% to about 1%, and more preferably from about 0.01% to about 0.5%; and from about 2% to about 90% of a topical pharmaceutically-acceptable emollient.

The term “emollients” includes materials used for the prevention or relief of dryness, as well as for the protection of the skin. A wide variety of suitable emollients are known and may be used herein. Sagarin, Cosmetics, Science and Technology, 2nd Edition, Vol. 1, p. 32-43 (1972), incorporated herein by reference, contains numerous examples of suitable materials.

A lotion can be made from a solution carrier system. Lotions preferably comprise from 0.0001% to about 10% of the active compound (e.g., a creatine compound), more preferably from about 0.001% to about 1%, and more preferably from about 0.01% to about 0.5%; from about 1% to about 20%, preferably from about 5% to about 10%, of an emollient; and from about 50% to about 90%, preferably from about 60% to about 80%, water.

Another type of product that may be formulated from a solution carrier system is a cream. A cream of the present invention would preferably comprise from about 0.0001% to about 10% of the active compound (e.g., a creatine compound), more preferably from about 0.001% to about 1%, and more preferably from about 0.01% to about 0.5% of the active compound; from about 5% to about 50%, preferably from about 10% to about 20%, of an emollient, and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble. An ointment may also comprise from about 2% to about 10% of an emollient plus from about 0.1% to about 2% of a thickening agent. A more complete disclosure of thickening agents useful herein can be found in Segarin, Cosmetics, Science and Technology, 2nd Edition, Vol. 1, pp. 72-73 (1972), incorporated herein by reference.

If the carrier is formulated as an emulsion, from about 1% to about 10%, preferably from about 2% to about 5%, of the carrier system comprises an emulsifier. Emulsifiers may be nonionic, anionic or cationic. Suitable emulsifiers are disclosed in, for example, U.S. Pat. No. 3,755,560; U.S. Pat. No. 4,421,769; and McCutcheon's Detergents and Emulsifiers, North American Edition, pages 317-324 (1986); the disclosures of which are incorporated herein by reference. Preferred emulsifiers are anionic or nonionic, although the other types may also be used.

Lotions and creams can be formulated as emulsions as well as solutions. Preferably such lotions comprise from about 0.0001% to about 10% of the active compound (e.g., a creatine compound), more preferably from about 0.001% to about 1%, and more preferably from about 0.01% to about 0.5% of the active compound; from about 1% to about 20%, preferably from about 5% to about 10%, of an emollient; from about 25% to about 75%, preferably from about 45% to about 95%, water; and from about 0.1% to about 10%, preferably from about 0.5% to about 5%, of an emulsifier. Such creams would preferably comprise from about 0.0001% to about 10% of the active compound (e.g., a creatine compound), more preferably from about 0.001% to about 1%, and more preferably from about 0.01% to about 0.5% of the active compound; from about 1% to about 20%, preferably from about 5% to about 10%, of an emollient; from about 20% to about 80%, preferably from about 30% to about 70%, water; and from about 1% to about 10%, preferably from about 2% to about 5%, of an emulsifier.

Single emulsion skin care preparations, such as lotions and creams, of the oil-in-water type and water-in-oil type are well-known in the cosmetic art and are useful in the present invention. Multiphase emulsion compositions, such as the water-in-oil-in-water type, as disclosed in U.S. Pat. No. 4,254,105, incorporated herein by reference, are also useful in the present invention. In general, such single or multiphase emulsions contain water, emollients and emulsifiers as essential ingredients.

Triple emulsion carrier systems comprising an oil-in-water-in-silicone fluid emulsion composition as disclosed in U.S. Pat. No. 4,960,764, incorporated herein by reference, are also useful in the present invention. Preferably, this triple emulsion carrier system can be combined with from about 0.0001% to about 10% of the active compound (e.g., a creatine compound), more preferably from about 0.001% to about 1%, and more preferably from about 0.01% to about 0.5% of the active compound to yield the topical pharmaceutical composition of the present invention.

Another emulsion carrier system useful in the topical pharmaceutical compositions of the present invention is a micro-emulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water. This carrier system is preferably combined with from about 0.0001% to about 10% of the active compound (e.g., a creatine compound), more preferably from about 0.001% to about 1%, and more preferably from about 0.01% to about 0.5% of the active compound.

If the topical pharmaceutical compositions of the present invention are formulated as a gel or a cosmetic stick, a suitable amount of a thickening agent, as disclosed supra, is added to a cream or lotion formulation.

The topical pharmaceutical compositions of the present invention may also be formulated as makeup products such as foundations.

The topical pharmaceutical compositions of the present invention may also be. formulated as medicated pads. Suitable examples of these pads are fully disclosed in U.S. Pat. Nos. 4,891,227 and 4,891,228, the disclosures of which are incorporated herein by reference.

The topical pharmaceutical compositions of the present invention may contain, in addition to the aforementioned components, a wide variety of additional oil-soluble materials and/or water-soluble materials conventionally used in topical compositions, at their art-established levels, such as, for example, acidulants, surfactants, emulsifiers, gelling agents, penetration enhancers, solubilizers, skin protectants, and sunscreen agents.

Various water-soluble materials may also be present in the compositions of this invention. These include humectants, proteins and polypeptides, preservatives and an alkaline agent. In addition, the topical compositions herein can contain conventional cosmetic adjuvants, such as dyes, opacifiers (e.g., titanium dioxide), pigments and perfumes.

The topical pharmaceutical compositions of the present invention may also include a safe and effective amount of a penetration enhancing agent. A preferred amount of penetration enhancing agent is from about 1% to about 5% of the composition. Another useful penetration enhancer for the present invention is the non-ionic polymer under the CTFA designation: polyacrylamide and isoparrafin and laureth-7, available as Sepigel from Seppic Corporation. Also useful is polyquatemium-32 and mineral oil known as SalCare SC92 available from Allied Colloids, Suffolk, Va. This is a class of cationic polymers which are generally described in U.S. Pat. No. 4,628,078 and U.S. Pat. No. 4,599,379, both of which are incorporated by reference herein.

Examples of useful penetration enhancers, among others, are disclosed in U.S. Pat. No. 4,537,776; U.S. Pat. No. 4,552,872; U.S. Pat. No. 4,557,934; U.S. Pat. No. 4,130,667; U.S. Pat. No. 3,989,816; U.S. Pat. No. 4,017,641; and European Patent Application 0043738, the contents of each of which are incorporated herein by reference.

Other conventional skin care product additives may also be included in the compositions of the present invention. For example, collagen, hyaluronic acid, elastin, hydrolysates, primrose oil, jojoba oil, epidermal growth factor, soybean saponins, mucopolysaccharides, and mixtures thereof may be used.

Various vitamins may also be included in the compositions of the present invention. For example, Vitamin A, ascorbic acid, Vitamin B, biotin, panthothenic acid, Vitamin D, Vitamin E and mixtures thereof and derivatives thereof are contemplated.

Also contemplated are skin cleaning compositions comprising both active compounds of the present invention and a cosmetically-acceptable surfactant. The term “cosmetically-acceptable surfactant” includes a surfactant which is not only an effective skin cleanser, but also can be used without undue toxicity, irritation, allergic response, and the like. Furthermore, the surfactant must be capable of being comingled with the active compound in a manner such that there is no interaction which would substantially reduce the efficacy of the composition.

The skin cleaning compositions of the present invention preferably contain from 0.0001% to about 10% of the active compound (e.g., a creatine compound), more preferably from about 0.001% to about 1%, and more preferably from about 0.01% to about 0.5% of the active compound and from about 1% to about 90%, more preferably from about 1% to about 10%, of a cosmetically-acceptable surfactant.

The physical form of the skin cleansing compositions is not critical. The compositions can be, for example, formulated as toilet bars, liquids, pastes, mousses, or pads.

The surfactant component of the compositions of the present invention are selected from anionic, nonionic, zwitterionic, amphoteric and ampholytic surfactants, as well as mixtures of these surfactants. Such surfactants are well-known to those skilled in the detergency art.

The cleaning compositions of the present invention can optionally contain, at their art-established levels, materials which are conventionally used in skin cleansing compositions.

Sunblocks and sunscreens incorporating creatine compounds are also contemplated. The term “sun block” or “sun screen” includes compositions which block UV light. Examples of sunblocks include, for example, zinc oxide and titanium dioxide.

The combination of creatine compounds with a UVA and/or UVB sunscreen would be advantageous. The inclusion of sunscreens in compositions of the present invention will provide immediate protection against acute UV damage.

A wide variety of conventional sunscreening agents are suitable for use in combination with the active compound. Segarin, et al., at Chapter VIII, pages 189 et seq., of Cosmetics Science and Technology, disclose numerous suitable agents. Specific suitable sunscreening agents include, for example: p-aminobenzoic acid, its salts and its derivatives (ethyl, isobutyl, glyceryl esters; p-dimethylaminobenzoic acid); anthranilates (i.e., o-aminobenzoates; methyl, menthyl, phenyl, benzyl, phenylethyl, linalyl, terpinyl, and cyclohexenyl esters); salicylates (amyl, phenyl, benzyl, menthyl, glyceryl, and dipropyleneglycol esters); cinnamic acid derivatives (methyl and benzyl esters, alpha-phenyl cinnamonitrile; butyl cinnamoyl pyruvate); dihydroxycinnamic acid derivatives (umbelliferone, methylumbelliferone, methylaceto-umbelliferone); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); dihydroxy-naphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); Hydroxy- or methoxy-substituted benzophenones; uric and vilouric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-iso-propyldibenzoylmethane; butylmethoxydibenzoylmethane; etocrylene; and 4-isopropyl-di-benzoylmethane.

Preferred sunscreens useful in the compositions of the present invention are 2-ethylhexyl-p-methoxycinnamate, butylmethoxydibenzoylmethane, 2-hydroxy-4-methoxybenzophenone, octyldimethyl-p-aminobenzoic acid and mixtures thereof.

A safe and effective amount of sunscreen may be used in the compositions of the present invention. The sunscreening agent must be compatible with the active compound. Generally the composition may comprise from about 1% to about 20%, preferably from about 2% to about 10%, of a sunscreening agent. Exact amounts will vary depending upon the sunscreen chosen and the desired Sun Protection Factor (SPF).

Also particularly useful in the present invention are sunscreens such as those disclosed in U.S. Pat. No. 4,937,370 and U.S. Pat. No. 4,999,186, incorporated herein by reference. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range.

An agent may also be added to any of the compositions of the present invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157 which is incorporated herein by reference.

In another embodiment of the present invention, an anti-inflammatory agent is included as an active agent along with the creatine compounds of the invention. The anti-inflammatory agent protects strongly in the UVA radiation range (though it also provides some UVB protection as well) thereby preventing further skin damage caused by UV radiation, while the creatine compounds of the invention treats existing damage. Thus the combination provides broad protection. The topical use of anti-inflammatory agents reduces photo-aging of the skin resulting from chronic exposure to UV radiation. (See U.S. Pat. No. 4,847,071 and U.S. Pat. No. 4,847,069 both of which are incorporated herein by reference.)

A safe and effective amount of an anti-inflammatory agent may be added to the compositions of the present invention, preferably from about 0.1% to about 10%, more preferably from about 0.5% to about 5%, of the composition. The exact amount of anti-inflammatory agent to be used in the compositions will depend on the particular anti-inflammatory agent utilized since such agents vary widely in potency.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.

A second class of anti-inflammatory agents which is useful in the compositions of the present invention includes the nonsteroidal anti-inflammatory agents. The variety of compounds encompassed by this group are well-known to those skilled in the art. For detailed disclosure of the chemical structure, synthesis, side effects, etc., of non-steroidal anti-inflammatory agents, reference may be had to standard texts, including Antiinflammatory and Anti-Rheumatic Drugs, K. D. Rainsford, Vol. I-III, CRC Press, Boca Raton, (1985), and Anti-inflammatory Agents. Chemistry and Pharmacology, 1, R. A. Scherrer, et al., Academic Press, New York (1974). Mixtures of these non-steroidal anti-inflammatory agents may also be employed, as well as the pharmaceutically-acceptable salts and esters of these agents. For example, etofenamate, a flufenamic acid derivative, is particularly useful for topical application. Yet another class of anti-inflammatory agents which are useful in the present invention are those disclosed in U.S. Pat. No. 4,912,248, incorporated herein by reference. This patent discloses compounds and diastereomeric mixtures of specific 2-naphthyl-containing ester compounds, especially naproxen ester and naproxol ester compounds, having two or more chiral centers. Finally, so-called “natural” anti-inflammatory agents are useful in the present invention. For example, candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), may be used.

In another embodiment, the skin composition further comprises a safe and effective amount of a skin protectant. The skin protectant preferably comprises from about 0.001% to about 2%, more preferably from about 0.01% to about 1% of the composition. Useful skin protectants are disclosed in the Federal Register Vol. 48, No. 32 and include allantoin, aluminum hydroxide gel, bismuth subnitrate, boric acid, calamine, cocoa butter, corn starch, dimethicone, glycerin, kaolin, live yeast cell derivative, petrolatum, shark liver oil, sodium bicarbonate, sulfur, tannic acid, white petrolatum, zinc acetate, zinc carbonate and zinc oxide and mixtures thereof.

As set out above, certain embodiments of the present compounds can contain a basic functional group, such as amino or alkylamino, and are, thus, capable of forming pharmaceutically acceptable salts with pharmaceutically acceptable acids. The term “pharmaceutically acceptable salts” in this respect, includes the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances includes the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “pharmaceutically acceptable esters” includes the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term is further intended to include lower hydrocarbon groups capable of being solvated under physiological conditions, e.g., alkyl esters, methyl, ethyl and propyl esters. (See, for example, Berge et al., supra.)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for topical or oral, administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect, as described herein.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The preparations of the present invention may be given topically or orally. They are of course given by forms suitable for each administration route. For example, they are administered in tablets or capsule form, ointment; topical by lotion or ointment. Topical administration is preferred.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

The term “chronic treatment” includes continued treatment with a creatine compound over an extended period during a subject's lifetime, preferably for at least about three weeks, more preferably from about three months to about twenty years, more preferably from about six months to about ten years, more preferably still from about one year to about five years.

While it is possible for a compound of the present invention to be administered alone, it is preferable to administer the compound as a pharmaceutical composition.

In a further embodiment, the invention contemplates co-administering to the subject an effective amount of a skin preserving agent. Examples of skin preserving agents include antioxidants, such as ascorbic acid, vitamins, coenzyme Q10 (CoQ10) and its derivatives, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. Preferred anti-oxidants include, CoQ10 and vitamin E. Other examples of skin preserving agents include energy-enhancing agents (e.g., ATP, nicotinamide or pyruvate), vitamins (e.g., E, C, B5, B6, and B9) and vitamin precursors.

The creatine compound can be administered to the afflicted individual alone or in combination with another creatine compound or other agent. The other agents could be approved therapies, supplements that protect against oxidative damage, energy enhancers, sugars, intermediates of metabolism and nutrients among others. The creatine compounds can be administered as pharmaceutically acceptable salts in a pharmaceutically acceptable carrier. The compound may be administered to the subject by a variety of routes, including, but not necessarily limited to topical, oral (dietary), transdermal, or parenteral (e.g., subcutaneous, intramuscular, intravenous injection, bolus or continuous infusion) routes of administration, for example. An effective amount (i.e., one that is sufficient to produce the desired effect in an individual) of a composition comprising a creatine analog is administered to the individual. The actual amount of the creatine compound to be administered will depend on factors such as the size and age of the individual, in addition to the severity of symptoms, other medical conditions and the desired aim of treatment. As discussed above, preferably the compound is administered topically.

The creatine compound can be formulated according to the selected route of administration (e.g., emulsion, solution, cream, powder, tablet, capsule, transdermal patch). An appropriate composition comprising a creatine analog can be prepared in a physiologically acceptable vehicle or carrier. For example, a composition in tablet form can include one or more additives such as a filler (e.g., lactose), a binder (e.g., gelatin, carboxymethylcellulose, gum arabic), a flavoring agent, a coloring agent, or coating material as desired. For solutions or emulsions in general, carriers may include aqueous or alcoholic/aqueous solutions, emulsions or suspensions, including saline and buffered media. Parenteral vehicles can include sodium chloride, solution, Ringer's dextrose, dextrose and sodium chloride, lactated Ringer's or fixed oils. In addition, intravenous vehicles can include fluid and nutrient replenishers, and electrolyte replenishers, such as those based on Ringer's dextrose. Preservatives and other additives can also be present. For example, antimicrobial, antioxidant, chelating agents, and inert gases can be added. (See, generally, Remington's Pharmaceutical Sciences, 16th Edition, Mack, Ed., 1980).

The term “administration” is intended to include routes of administration which allow the creatine compounds to perform their intended function. Examples of routes of administration which may be used include injection (topical, oral, subcutaneous, intravenous, parenterally, intraperitoneally, inhalation, transdermal, and rectal. Depending on the route of administration, the creatine compound may be coated with or in a material to protect it from the natural conditions which may detrimentally effect its ability to perform its intended function. The administration of the creatine compound is done at dosages and for periods of time effective to reduce, ameliorate or eliminate the symptoms of aging. Dosage regimes may be adjusted for purposes of improving the therapeutic or prophylactic response of the compound. For example, several divided doses may be administered daily or the dose may be proportionally reduced as indicated by the exigencies of the therapeutic situation.

In one embodiment, the invention pertains, at least in part to a packaged composition comprising an effective amount of a creatine compound, a cosmetically acceptable carrier, and one or more cosmetic adjuvants. In one embodiment, the effective amount of the creatine compound is at least between about 0.0001 and 10% by weight, more preferably between about 0.001 and 1% by weight, and even more preferably between about 0.01 and 0.5% by weight.

In another embodiment, the packaged composition further comprises instructions for use. In one embodiment, the instructions for use are instructions for all the methods described herein.

### EXEMPLIFICATION OF THE INVENTION

### Example 1

### Effect of Creatine Ascorbate, Creatine Monohydrate and Creatine Pyruvate on Lipid Peroxidation

Lipid peroxidation (the oxidative breakdown of polyunsaturated fatty acids) is widely accepted as one of the general mechanisms of cellular injury and death. The purpose of this example was to assess the ability of creatine ascorbate, creatine monohydrate and creatine pyruvate to function as free radical scavengers by measuring their inhibitory activity against UV-induced lipid peroxidation, as compared to ascorbic acid and magnesium ascorbyl phosphate.

### Methods

Test materials were added to dispersions of lecithin (a natural phospholipid) and irradiated with UVB light (11,000 μW/cm² at the source). After 3 hours, trichloroacetic acid and thibarbituric acids were added and the concentration of Thiobarbituric Acid Reactive Substance (TBARS), such as malondialdehyde, was measured spectroscopically (malonaldehyde is a breakdown product generated spontaneously from oxidized lipid) at 550 nm using an UV microplate reader. For references describing this method, see Pelle et al. “Arch. Biochem. Biophys.” 283:234-40 (1990); Wang et al. “J. Ethnopharmacol.” 82:169-175 (2002).

### Results

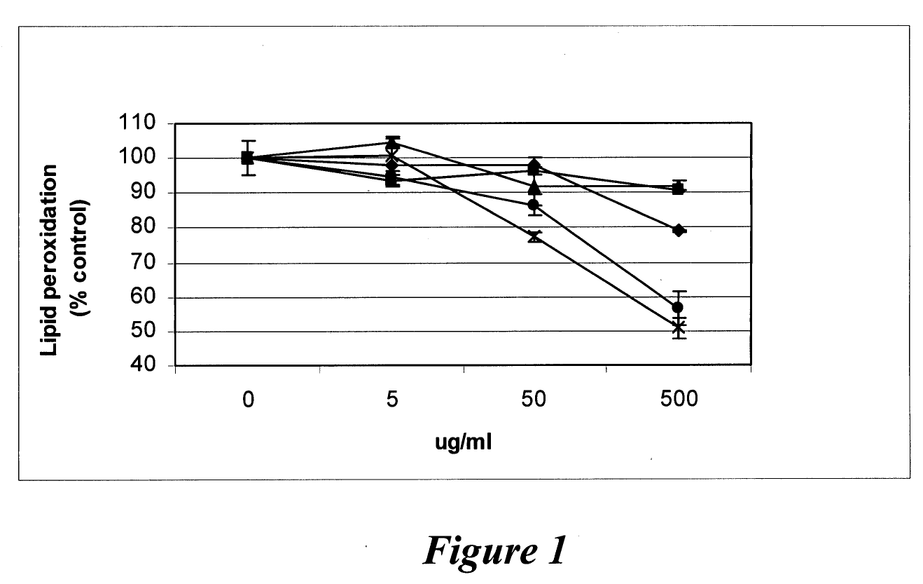

FIG. 1 is a graph which illustrates the concentration dependent effects of several creatine compounds, as well as ascorbic acid and magnesium ascorbyl phosphate, on the inhibition of lipid peroxidation. FIG. 1 indicates that creatine ascorbate (♦) exhibited the best inhibition of lipid peroxidation (21% inhibition at 500 μg/mL) when compared to the other creatine compounds tested. Creatine monohydrate (▪) and creatine pyruvate (▴) exhibited less than 10% inhibitory activity at all concentrations tested. Both ascorbic acid and magnesium ascorbyl phosphate had good inhibitory activity (up to 49%), which demonstrated the technical success of the assay.

### Example 2

### Effect of Creatine Ascorbate, Creatine Monohydrate and Creatine Pyruvate on Type I Collagen in Human Dermal Fibroblast Conditioned Medium

Collagen secreted by dermal fibroblasts is a major component of the extracellular matrix in the skin. In aged and photodamaged skin, the level of new collagen is decreased due to the lower number and deregulation of dermal fibroblasts. The purpose of this example was to test creatine ascorbate (CA), creatine monohydrate (CM) and creatine pyruvate (CP) on type I collagen levels in human dermal fibroblast conditioned medium as compared with ascorbic acid (AA) and magnesium ascorbyl phosphate (MAP).

### Methods

Normal human dermal fibroblasts (passage 5, lot number 7F1245, Cambrix, Walkersville, Md.) were seeded in a 96-well plate in DMEM medium (high glucose) containing 5% fetal calf serum and grown to late subconfluent stage. Two sets of aqueous solutions of 10 mg/mL of creatine ascorbate, creatine monohydrate, creatine pyruvate, ascorbic acid and magnesium ascorbyl phosphate were prepared in Type I water. The first set of solutions were prepared immediately before being added to cell cultures. The second set of solutions were preincubated at pH 4.0 (+0.1) five days, then the solutions were lyophilized and the substrates were redissolved in water at 10 mg/mL prior to being added to the cell cultures. Both the freshly prepared solutions and the solutions of preincubated test materials were administered to the cells, and cell culture conditioned media was harvested 5 days after the start of the experiment and tested for type I collagen by sandwich ELISA using affinity purified antibodies, followed by streptavidin-avidin-HRP conjugate and ABTS, according to standard ELISA protocol. The colorimetric signal proportional to collagen content was measured with a microplate spectrophotometer at 405 nm. For references describing this method, see Dobek et al. “J. Dermatol. Sci.” 8:18 (1994) and Zhao et al. “Phytomedicine” 12:132 (2005).

### Results

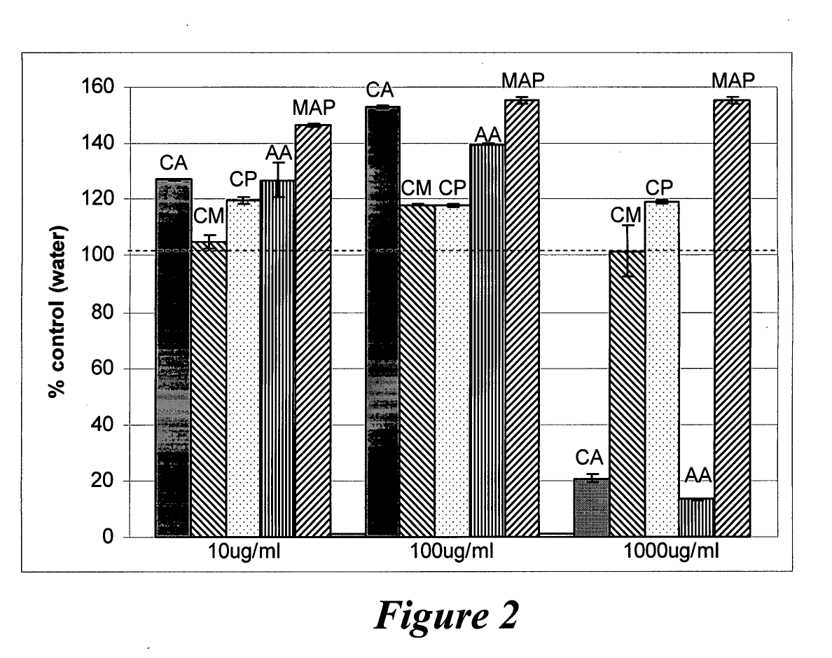

FIG. 2 illustrates the concentration-dependent effect of freshly prepared test materials on the stimulation of type I collagen in human dermal fibroblast conditioned medium. The control is shown as the dashed line at 100%. As expected, there was a large increase in the levels of type I collagen in the cell cultures exposed to all concentrations of magnesium ascorbyl phosphate (MAP). Creatine ascorbate also significantly increased the levels of type I collagen at concentrations of 10 μg/mL and 100 μg/mL, although the amount of type I collagen expressed at the highest concentration was significantly lower. Creatine monohydrate (CM) and creatine pyruvate (CP) both moderately increased type I collagen levels at concentrations of 10 μg/mL and 100 μg/mL, although at 1000 μg/mL, the creatine monohydrate and creatine pyruvate both were better than creatine ascorbate and ascorbic acid:

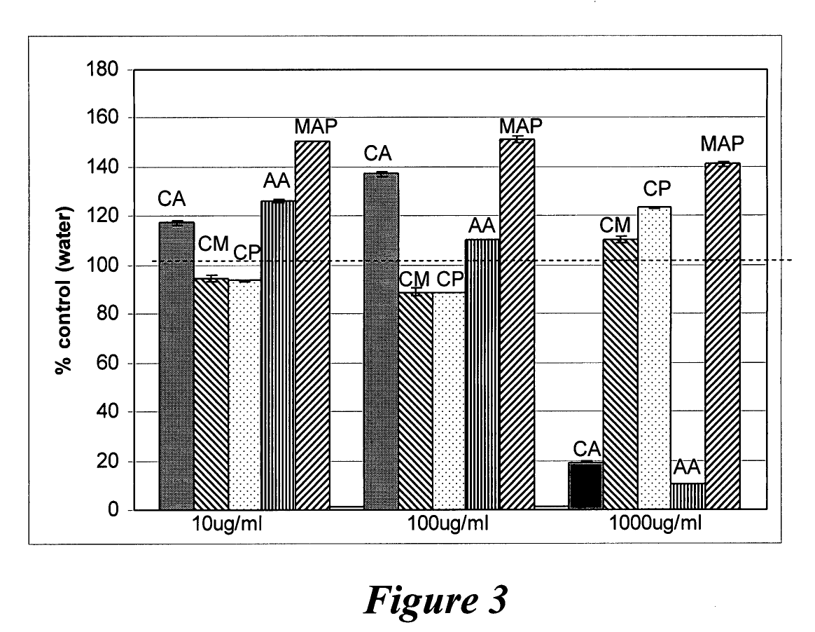

FIG. 3 illustrates the concentration-dependent effect of preincubated test materials on the stimulation of type I collagen in human dermal fibroblast conditioned medium. The results are similar to those shown in FIG. 3. The control is shown as the dashed line at 100%.

In conclusion, the results of both assays demonstrated the strong stimulation of type I collagen by magnesium ascorbyl phosphate which indicated the technical success of the experiment. Creatine ascorbate showed a dose-dependent stimulation of type I collagen levels by about 50% above the non-treated control (water). This stimulation was comparable to that of magnesium ascorbyl phosphate and better than ascorbic acid in the 100 μg/mL concentration.

### Example 3

### Effect of Creatine Ascorbic, Creatine Monohydrate and Creatine Pyruvate on Mitochondrial Metabolism in Human Dermal Fibroblast Cultures

The purpose of this assay was to determine the effect creatine ascorbate, creatine monohydrate and creatine pyruvate on the mitochondrial metabolism on the entire cell culture using an MTT assay. The MTT assay measures the activity of succinate dehydrogenase, a key enzyme in the respiratory electron transport chain in mitochondia.

### Methods

Two sets of aqueous solutions of 10 mg/mL of creatine ascorbate, creatine monohydrate, creatine pyruvate, ascorbic acid and magnesium ascorbyl phosphate were prepared in Type I water. The first set of solutions were prepared immediately before being added to cell cultures. The second set of solutions were preincubated at pH 4.0 (+0.1) five days, then the solutions were lyophilized and the substrates were redissolved in water at 10 mg/mL prior to being added to the cell cultures. Normal human dermal fibroblasts (passage 5, lot number 7F1254, Cambrex, Walkersville, Md.) were seeded in a 96-well plate in phenol red-free DMEM medium (high glucose) containing 2.5% fetal calf serum at 2500 cells per well and test materials, both freshly prepared solutions and preincubated solutions, were added 24 hours later. At the end of the incubation period MTT (3-(4,5-dimethylthiazol-2-yl)-2,5-diphenyltetrazolium bromide) was added to the cell cultures and incubation was pursued for an additional three hours. Culture media were then discarded, the cells were rinsed and the intracellular MTT reduction product, formazan, was solubilized in ethanol/acetic acid. The colorimetric signal proportional to the mitochondrial activity in the cell cultures was measured with a microplate spectrophotometer at 570 nm. For references describing this method, see Berridge et al. “Arch. Biochem. Biophys.” 303:474-482 (1993) and Murimaki et al. “Biochem. Pharmacol.” 44:2191-2197 (1992).

### Results

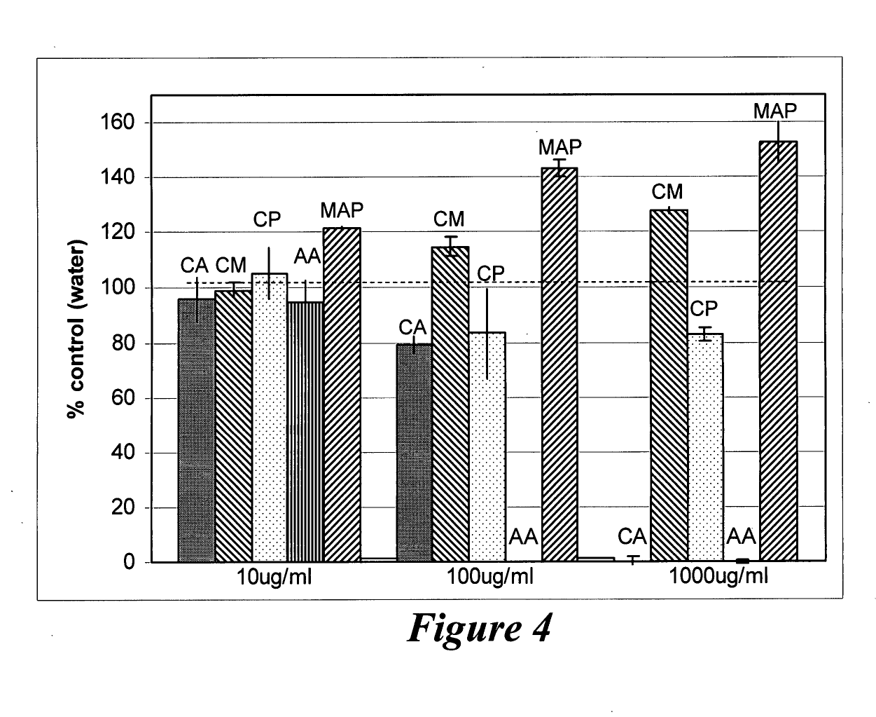

FIG. 4 illustrates the concentration-dependent effect of freshly prepared creatine ascorbate (CA), creatine monohydrate (CM), creatine pyruvate (CP), ascorbic acid (AA) and magnesium ascorbyl phosphate (MAP) solutions on the stimulation of mitochondrial metabolism in human dermal fibroblasts. The control is shown as the dashed line at 100%. Freshly prepared magnesium ascorbyl phosphate exhibited the greatest stimulatory activity on the mitochondrial metabolism in human dermal fibroblasts, which illustrates the technical success of this assay. Freshly prepared creatine monohydrate stimulated the mitochondrial metabolism in a concentration-dependent manner (0% stimulation at 10 μg/mL, 15% stimulation at 100 μg/mL and 28% stimulation at 0.1000 μg/mL). Freshly prepared creatine pyruvate inhibited mitochondrial metabolism (17% at 100 μg/mL and 11000 μg/mL), while freshly prepared creatine ascorbate and ascorbic acid did not have a stimulatory effect at any concentration tested and inhibited mitochondrial metabolism at 100 μg/mL and 1000 μg/mL.

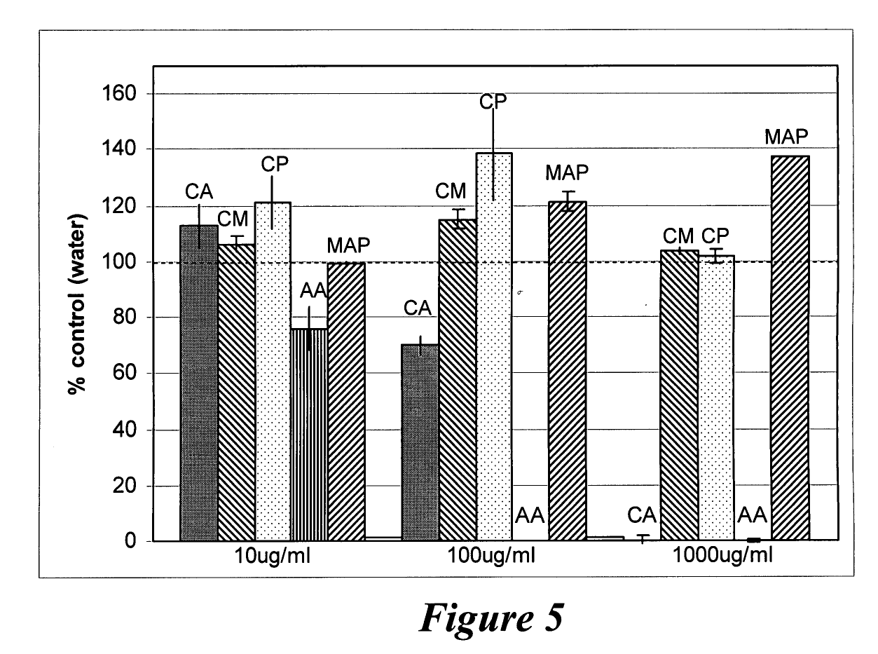

FIG. 5 illustrates the concentration-dependent effect of preincubated creatine ascorbate (CA), creatine monohydrate (CM), creatine pyruvate (CP), ascorbic acid (AA) and magnesium ascorbyl phosphate (MAP) solutions on the stimulation of mitochondrial metabolism in human dermal fibroblasts. The preincubated magnesium ascorbyl phosphate also exhibited stimulation of mitochondrial metabolism in human dermal fibroblasts. Preincubated creatine monohydrate stimulated mitochondrial metabolism, although not to the extent of the freshly prepared creatine monohydrate. Preincubated creatine pyruvate was not to be inhibitory, although the freshly prepared creatine pyruvate was inhibitory. Preincubated creatine ascorbate was superior to preincubated ascorbic acid, which had a 24% inhibitory effect on mitochondrial metabolism, at the same concentration.

### Example 4

### Effect of Creatine Ascorbate Creatine Monohydrate and Creatine Pyruvate as Antioxidants

Because of their propensity to damage to vital biological systems, reactive species, such as free radicals, have been implicated in aging and in more than 100 disease. Production of oxygen reactive species is an integral part of human metabolism is the skin. Furthermore, production of oxygen reactive species is increased upon exposure to sunlight, pollutants and during inflammation. The objective of this test was to measure the antioxidant potential of creatine ascorbate, creatine phosphate and creatine monohydrate using oxygen radical absorbance capacity (ORAC) assay.

### Methods

The ORAC assay was performed according to the method described by Ou et al. “J. Agric. Food Chem.” 49(10):4619-4626 (2001). The ORAC assay measured the ability of antioxidant components to inhibit the decline in disodium fluorescein (FL) fluorescence that is induced by the peroxyl radical generator 2′,2′-Azobis(2-amidinopropane)dihydrochloride (AAPH). The reaction was conducted in a 96-well plate format. The reaction mixture contained FL (6.3×10⁻⁷ M) and AAPH (1.28×10⁻¹ M) in phosphate-buffered saline (PBS). The final dilution of test materials added (stock solutions at 1000 μg/mL, 100 μg/mL and 10 μg/mL) was 1/20. The reaction was started by the addition of AAPH. Fluorescence was measured at the emission wavelength of 530 nm and excitation wavelength of 485 nm using a microplate fluorimeter. ORAC values were calculated from the quantitation of the areas under the FL decay curve and are expressed as micromole μmol Trolox equivalents (TE) per mL.

### Results

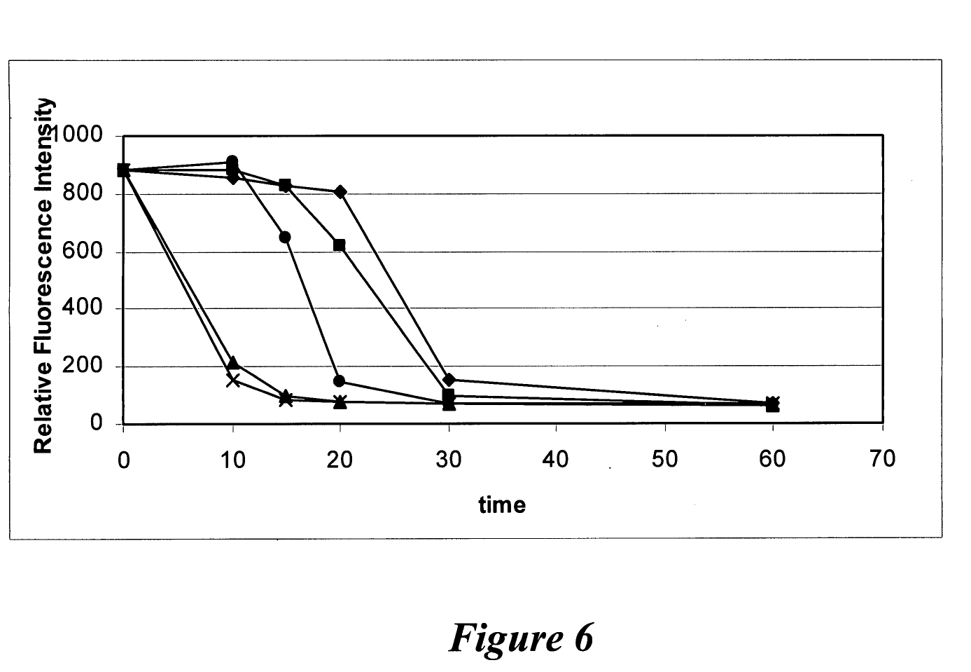

FIG. 6 shows the relative fluorescence intensity of creatine ascorbate (▪), creatine monohydrate (▴), creatine pyruvate (×), ascorbic acid (♦) and magnesium ascorbyl phosphate (•) over 60 minutes. This data indicates that creatine ascorbate has an antioxidant effect similar to that of ascorbic acid and greater than magnesium ascorbyl phosphate.

### Example 5

### Effect of Creatine Monohydrate, Creatine Pyruvate and Creatine Ascorbate on p53 Protein Expression

The objective of this example was to determine whether creatine monohydrate, creatine pyruvate and creatine ascorbate have an effect on the expression of p53 in human dermal fibroblasts. P53 is a key protein triggering genomic repair and apoptosis (programmed cell death) in response to mutagenic stress such as UV irradiation and carcinogens.

### Methods

### A. Determination of Irradiation Dose

Human dermal fibroblasts were grown to subconfluence in phenol-free DMEM medium (4 g/L glucose) supplemented with 5% serum and irradiated with UVB light at 0.15 mW/cm² for various periods of time. Cells were than returned to incubator for 72 hours and cell numbers were determined by sulforhodamine B method. For a description of this assay, see Skehan et al. “J. Natl. Cancer Inst.” 82:1107 (1990).

### B. Effect of Test Materials on P53 Induction by UVB Irradiation

Confluent human dermal fibroblasts were trypsinized, counted and plated at 750,000 cells/well in eight 6-well plates in phenol-free DMEM medium (4 g/L glucose) supplemented with 5% serum. After three hours, creatine ascorbate, creatine monohydrate, creatine pyruvate, ascorbic acid and magnesium ascorbyl phosphate in a concentration of 50 μg/ml were added and cells were incubated overnight at 37° C. in 5% CO₂ atmosphere. After 24 hours, p53 protein was induced by UVB irradiation at 0.15 mW/cm² for 120 and was allowed to accumulate for 4 hours. The cells were then lyzed and total p53 was quantified by sandwich ELISA. The p53 values were standardized with regard to total protein values determined with Bradford reagent. For a description of this assay, see Carlisle et al. “Toxicological Sciences” 55:60 (2000).

### Results

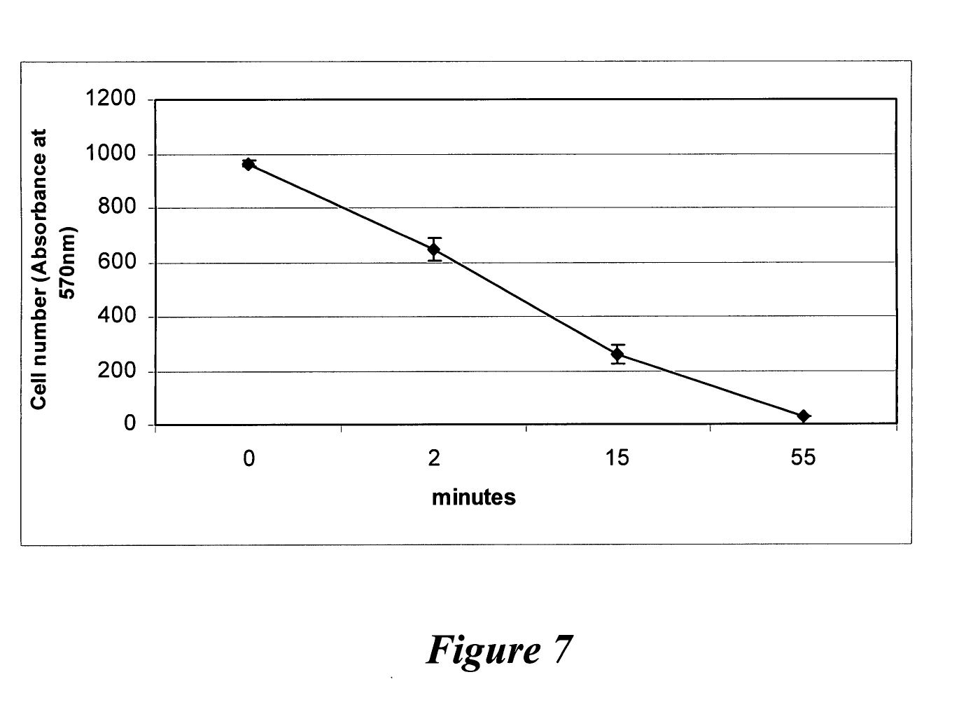

FIG. 7 illustrates the time (and thus dose) dependant effect of UVB irradiation at 0.15 mW/cm² on human dermal fibroblast proliferation. This data indicated that after 55 minutes, the number of cells decreases in a linear manner and that the number of cells decreased by about 35%. A 35% reduction is indicative of genomic damage strong enough to trigger p53 upregulation, without compromising the cultures.

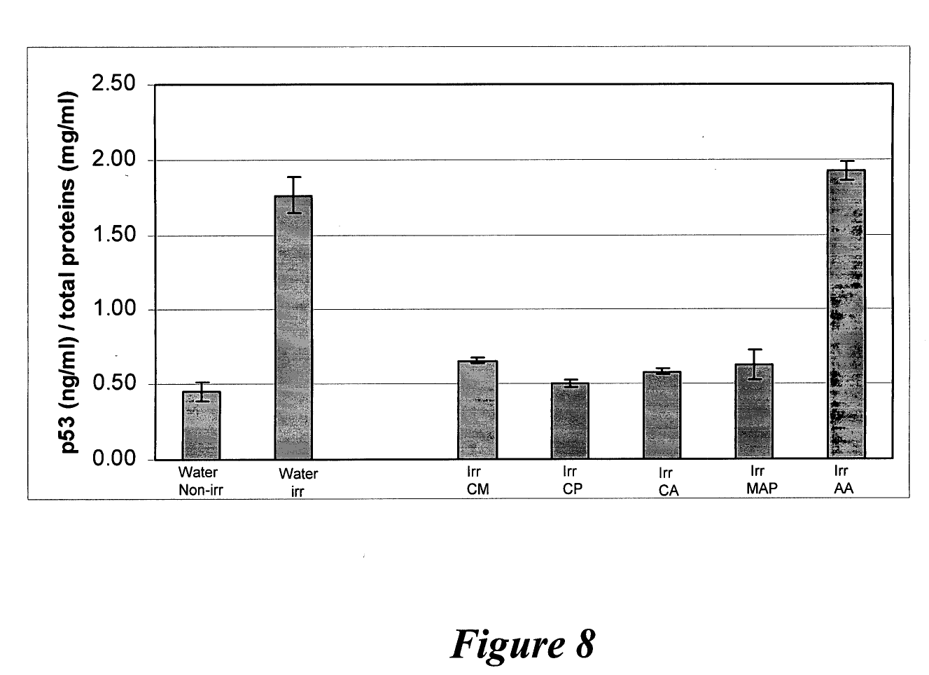

FIG. 8 illustrates the effect of creatine monohydrate (CM Irr), creatine ascorbate (CA Irr), creatine pyruvate (CP Irr), ascorbic acid (AA Irr) and magnesium ascorbyl phosphate (MAP Irr) on p53 expression induced by UVB irradiation of human dermal fibroblasts compared to non-irradiated and irradiated control cells. As expected the expression of p53 was low in non-irradiated fibroblasts (normal, healthy cells). Upon irradiation with UVB radiation, the p53 protein level increased, as expected, within four hours. Ascorbic acid showed no effect on inhibiting p53 expression by the cells upon irradiation. However, p53 expression was significantly inhibited by creatine monohydrate, creatine pyruvate, creatine ascorbate and magnesium ascorbyl phosphate.

### Example 6

### Effect of Creatine Ascorbate, Creatine Monohydrate and Creatine Pyruvate on Metalloproteinase Activity

The objective of this example was to determine the effect of creatine ascorbate, creatine monohydrate and creatine pyruvate on metalloproteinase activity, compared with ascorbic acid and magnesium ascorbyl phosphate.

### Methods

Metalloproteinase (collagenase) activity was measured with Enzcheck kit from Molecular Probes using quenched fluorescent gelatin and Clostridium collagenase IV, a generic collagenase. Creatine ascorbate, creatine monohydrate, creatine pyruvate, ascorbic acid and magnesium ascorbyl phosphate, in concentrations of 1000 μg/mL, 100 μg/mL and 10 μg/mL, were incubated in the presence of a collagenase substrate (quenched fluorescing-linked gelatin) and in the presence of the proteolytic enzyme. Phenantroline, a potent metalloproteinase inhibitor was used as a positive control. The kinetics of the release of the digested, fluorescent gelatin were measured at excitation/emission wavelengths of 485/530 nm.

### Results

After incubation of 60 minutes, creatine ascorbate inhibited collagenase activity by 11.5% compared to the control, creatine pyruvate inhibited collagenase activity by 27% compared to the control and ascorbic acid inhibited collagenase activity by 39% when compared to the control. Neither creatine monohydrate nor magnesium ascorbyl phosphate showed any inhibitory activity.

### Example 7

### Effect of Creatine Ascorbate, Creatine Monohydrate and Creatine Pyruvate on Tyrosinase Activity

Tyrosinase is believed to be a key enzyme in the melanin synthesis pathway. The objective of this example was to assess the effect of creatine ascorbate, creatine monohydrate and creatine pyruvate on tyrosinase activity in vitro as compared with ascorbic acid and magnesium ascorbyl phosphate.

### Methods

Tyrosinase activity was measured according to the method described by Pomerantz “Biochem. Biophys. Res. Commun.” 16(2):188-194 (1964). Mushroom tyrosinase stock solution was prepared in PBS at 2000 U/mL and stored at 20° C. in 1 mL aliquots. Final concentration was 5 U/well (25 U/mL). Creatine ascorbate, creatine monohydrate, creatine pyruvate, ascorbic acid and magnesium ascorbyl phosphate were dissolved in PBS and further dilutions were made with Type I water. L-Dopa stock solution was 20 mM. The tyrosinase substrate was prepared in PBS. The assays were performed in a 96-well microtiter plates and read at 490 nm.

### Results

The results indicated that creatine ascorbate exhibited a significant inhibitory effect on the tyrosinase activity at concentrations of 500 μg/mL and 50 μg/mL. At 5 μg/mL, inhibition by creatine ascorbate was 26% compared to the control. Creatine ascorbate inhibitory activity was lower than the activity of ascorbic acid and was similar to the activity of magnesium ascorbyl phosphate. Creatine pyruvate displayed a concentration dependent inhibitory effect, with 100% inhibition at 500 μg/mL, 10% inhibition at 50 μg/mL and no inhibition at 5 μg/mL.

### Example 8

### Stability of Creatine Ascorbate, Creatine Monohydrate and Creatine Pyruvate

The objective of this example was to compare the stability of aqueous solutions of creatine ascorbate, creatine monohydrate and creatine pyruvate, compared with ascorbic acid and magnesium ascorbyl phosphate.

### Methods

Aqueous solutions of 10 mg/mL of creatine ascorbate, creatine monohydrate, creatine pyruvate, ascorbic acid and magnesium ascorbyl phosphate were prepared in Type I water and brought to pH 4.0 (±0.1) by adding acetic acid (creatine ascorbate, creatine monohydrate and magnesium ascorbyl phosphate) or sodium hydroxide (ascorbic acid and creatine pyruvate). The solutions were incubated for 5 days at room temperature and were subsequently lyophilized, redissolved within 60 minutes of analysis and analyzed by isocratic HPLC on a C18 RF column (250 mm×4.6 mm) at 1 mL/min. The chromatography conditions are summarized in Table 1.

TABLE 1

| | | Detection |
|---|---|---|
| Compound | Mobile Phase | Wavelength (nm) |
| Creatine Ascorbate | Water | 245 |
| Creatine | 10% PBS | 210 |
| Monohydrate | | |
| Creatine Pyruvate | 10% PBS | 200 |
| Ascorbic Acid | 50% 50 mM Sulfuric acid | 245 |
| | 50% MeOH | |
| Magnesium | Water | 245 |
| Ascorbyl | | |
| Phosphate | | |

### Results

Creatine ascorbate and magnesium ascorbyl phosphate showed minor to no degradation. Creatine monohydrate showed a clear degradation product, and creatine pyruvate showed a 42% loss of the absorbance peak. Ascorbic acid also showed degradation as indicated by a 28% decrease of absorption.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

1. A method for treating uneven pigmentation in a subject's skin, comprising administering to said subject an amount of a creatine compound effective to modulate tyrosinase, wherein said amount of the creatine compound is at least between about 0.0001-10% by weight, such that said subject's skin is treated for uneven pigmentation.

2. The method of claim 1, wherein said creatine compound is an inhibitor of tyrosinase.

3. The method of claim 2, wherein said inhibitor inhibits tyrosinase by at least about 25% at a concentration of about 5 μg/mL of said creatine compound.

4. The method of claim 3, wherein said creatine compound is creatine ascorbate.

5. The method of claim 2, wherein said inhibitor inhibits tyrosinase by at least about 100% at a concentration of about 500 g/mL of said creatine compound.

6. The method of claim 2, wherein said inhibitor inhibits tyrosinase by at least about 10% at a concentration of about 50 g/mL of said creatine compound.

7. The method of claims 5 or 6, wherein said creatine compound is creatine pyruvate.

8. A method for quenching free radicals in a subject's skin, comprising administering to said subject an amount of a creatine compound effective to provide an antioxidant effect, wherein said amount of the creatine compound is at least between about 0.0001 and 10% by weight, such that free radicals are quenched.

9. The method of claim 8, wherein said creatine compound exhibits 5300 M Trolex equivalents/gram.

10. The method of claim 8, wherein said creatine compound exhibits antioxidant activity greater than magnesium ascorbyl phosphate (MAP).

11. The method of claims 9 or 10, wherein said creatine compound is creatine ascorbate.

12. A method for preserving a subject's skin, comprising administering to said subject an amount of a creatine compound effective to inhibit lipid peroxidation, wherein said amount of the creatine compound is at least about between about 0.0001 and 10% by weight, such that said subject's skin is preserved.

13. The method of claim 11, wherein said creatine compound inhibits by at least about 20% of lipid peroxidation at a concentration of about 500 g/mL of said creatine compound.

14. The method of claim 13, wherein said creatine compound is creatine ascorbate.

15. A method for treating aging of a subject's skin, comprising administering to said subject an amount of a creatine compound effective to modulate mitochondrial metabolism, wherein said amount of the creatine compound is at least between about 0.0001 and 10% by weight, such that said aging of a subject's skin is treated.

16. The method of claim 15, wherein said creatine compound stimulates mitochondrial metabolism.

17. The method of claim 16, wherein said creatine compound stimulates mitochondrial metabolism by at least about 15% at a concentration of about 100 g/mL of the creatine compound.

18. The method of claim 15, wherein said creatine compound stimulates mitochondrial metabolism by at least about 25% at a concentration of about 1000 g/mL of the creatine compound.

19. The method of claims 16 or 17, wherein said creatine compound is creatine monohydrate.

20. A method of treating a subject's skin for UV irradiation stress, comprising administering an effective amount of a creatine compound to said subject, wherein said effective amount of a creatine compound is a least between about 0.0001 and 10% by weight, such that the subject's skin is treated.

21. A method of modulating carcinogenic stress in a subject's skin, comprising administering an effective amount of a creatine compound to said subject, such that said carcinogenic stress is modulated.

22. The method of claim 21, wherein said creatine compound protects against or inhibits carcinogenic stress.

23. The method of claim 20 or 21, wherein said creatine compound is creatine monohydrate, creatine pyruvate or creatine ascorbate.

24. A method for modulating collagen levels in a subject's skin, comprising administering to said subject of a at least between about 0.0001 and 10% by weight of a creatine compound, such that collagen levels in said subject's skin are modulated.

25. The method of claim 24, wherein said creatine compound is elevates collagen levels.

26. The method of claim 25, wherein said creatine compound stimulates collagen by at least about 50% at concentration of about 10 μg/mL of the creatine compound.

27. The method of claim 26, wherein said creatine compound is creatine ascorbate.

28. The method of claim 25, wherein said creatine compound stimulated collagen by at least about 20% at a concentration of about 1 μg/mL of the creatine compound.

29. The method of claim 28, wherein said creatine compound is creatine pyruvate.

30. A method of treating inflammation in a subject, comprising administering an amount of a creatine compound effective to modulate metalloproteinase, such that said inflammation is treated.

31. The method of claim 30, wherein said creatine compound inhibits metalloproteinase.

32. The method of claim 31, wherein said metalloproteinase is collagenase.

33. The method of claim 31, wherein said creatine compound inhibits metalloproteinase by at least about 10% at a concentration of about 1 mg/mL of said creatine compound.

34. The method of claim 33, wherein said creatine compound is creatine ascorbate.

35. The method of claim 31, wherein said creatine compound inhibits metalloproteinase by at least about 25% at a concentration of about 1 mg/mL of said creatine compound.

36. The method of claim 35, wherein said creatine compound is creatine pyruvate.

37. The method of any one of claims 1-36, wherein said creatine compound is administered topically or orally.

38. The method of any one of claims 1-36, wherein said creatine compound is administered with a pharmaceutically acceptable carrier.

39. The method of claim 38, wherein said pharmaceutically acceptable carrier is suitable for administration as a liquid, solid, gelatin capsule, lotion, cream, mousse, aerosol and non-aerosol spray, gel, emulsion, solution, ointment, patch or medicated pad.

40. A cosmetic composition comprising an effective amount of a creatine compound, a cosmetically acceptable carrier, and one or more cosmetic adjuvants, wherein said effective amount of a creatine compound is at least about between about 0.0001-10% by weight.

41. The composition of claim 40, wherein said creatine compound is selected from the group consisting of creatine monohydrate, creatine ascorbate and creatine pyruvate.

42. The composition of claim 41, wherein said cosmetic composition is formulated as a foundation.

43. The composition of claim 40, wherein said cosmetic composition further comprises humectants, proteins, polypeptides, preservatives, acidulants or alkaline agents.

44. The composition of claim 41, wherein said effective amount is effective for preservation of skin, treating oxidation, inflammation, UV irradiation, photodamage, wrinkles, dryness, and skin aging.

45. The composition of claim 41, wherein said cosmetic adjuvant is a dye, opacifier, pigment, or a perfume.

46. The composition of claim 41, further comprising surfactants, emulsifiers, gelling agents, penetration enhancers, solubilizers, skin protectants, and sunscreen agents 47. A packaged composition comprising an effective amount of a creatine compound, a cosmetically acceptable carrier, and one or more cosmetic adjuvants, wherein said effective amount of a creatine compound is at least between about 0.0001 and 10% by weight.

48. The packaged composition of claim 47, further comprising instructions for use.

49. The method of any one of claims 1, 8, 12, 15, 20, and 24 wherein said effective amount is at least between about 0.001 and 1% by weight.

50. The method of any one of claims 1, 8, 12, 15, 20 and 24, wherein said effective amount is at least between about 0.01 and 0.5% by weight.

51. The composition of claim 40, wherein said effective amount is at least between about 0.001 and 1% by weight.

52. The composition of claim 40, wherein said effective amount is at least between about 0.01 and 0.5% by weight.

53. The packaged composition of claim 47, wherein said effective amount is at least between about 0.001 and 1% by weight.

54. The packaged composition of claim 47, wherein said effective amount is at least between about 0.01 and 0.5% by weight.
